(19) United States
(12) Patent Application Publication
(10) Pub. No.: US 2013/0331593 A1
(43) Pub. Date: Dec. 12, 2013

(54) Synthesis Of Treprostinil And Intermediates Useful Therein

(71) Applicants: Graham Mcgowan, Toronto (CA); Walter Giust, Toronto (CA); Danielle Marie Di Donato, Hamilton (CA); Teng-Ko Ngooi, Mississauga (CA); Jan Oudenes, Aurora (CA)

(72) Inventors: Graham Mcgowan, Toronto (CA); Walter Giust, Toronto (CA); Danielle Marie Di Donato, Hamilton (CA); Teng-Ko Ngooi, Mississauga (CA); Jan Oudenes, Aurora (CA)

(73) Assignee: ALPHORA RESEARCH INC., Mississauga, ON (CA)

(21) Appl. No.: 13/811,301

(22) PCT Filed: Jul. 22, 2011
(86) PCT No.: PCT/CA2011/050448

§ 371 Date: Aug. 30, 2013

(30) Foreign Application Priority Data
Jul. 22, 2010 (CA) 2,710,726

(51) Int. Cl. C07C 51/09; C07C 43/23; C07C 49/84; C07F 7/18

(52) U.S. Cl. CPC C07C 51/09; C07F 7/1892; C07C 43/23; C07C 49/84
USPC 556/436; 560/56; 568/654; 568/308

(57) ABSTRACT

Treprostinil is prepared by a process which involves Pauson-Khan cyclization of an an alkene-substituted, alkyne-substituted benzene corresponding to formula: (I) where PMB represents para-methoxy benzyl protecting group and R₁ and R₂ are alcohol protecting groups. Following cyclization, the resulting compound can be subjected to several chemical trans-formations followed by alkylation, hydrolysis and salt formation to yield treprostinil sodium. The use of para-methoxybenzyl group as the phenolic protecting group confers several process advantages that result in simplified purification of the final product and improved yields.

### FIELD OF THE INVENTION

This invention relates to a novel synthesis of the prostacyclin derivative treprostinil and intermediates useful in such syntheses.

### BACKGROUND OF THE INVENTION AND PRIOR ART

Prostacyclin derivatives are naturally occurring pharmaceutically active compounds, with a variety of pharmacological properties and utilities. A specific example of such prostacyclin derivative is treprostinil, which has the structural formula depicted below:

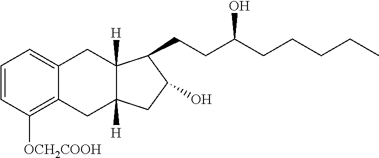

Treprostinil sodium, under the trade name Remodulin is indicated for oral use in management of pulmonary arterial hypertension in human patients. Other salt forms are proposed for administration by inhalation.

Treprostinil synthesis has previously been described in U.S. Pat. Nos. 6,700,025; 6,765,117; and 6,809,223; and Moriarty et. al., J. Org. Chem., 2004, 69, 1890-1902. The key step in these prior art syntheses is the Pauson-Khand “enyne cyclization” to complete the required tricyclic carbon skeleton and to install the required stereochemistry of the carbon skeleton. A Pauson-Khand enyne cyclization is the formal [2+2+1] cycloaddition of an alkene, an alkyne and carbon monoxide (usually provided in the form of a cobalt-CO complex) to form cyclopentenones. This Pauson-Khand reaction in treprostinil synthesis can be represented thus:

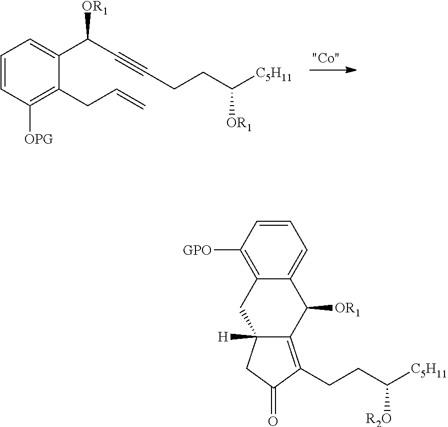

where PG is a protecting group such as methyl, cyanoalkyl, alkoxy, benzyl, tetrahydropyran (THP) or tert-butyldimethylsilyl (TBDMS), and R₁ and R₂ are alcohol protecting groups, for example tert-butyl silyl (TBS), TBDMS, THP or benzyl (Bn).

The choice of protecting group in such organic syntheses is based largely on their protection and deprotection efficacies at the desired stages. However, the protecting groups shown for use in treprostinil synthesis in these prior art items require different conditions for deprotection. This leads to an increase in the number of required deprotection steps, with added complications and reduced cost efficiency of the overall process.

It is an object of the present invention to provide a novel synthesis of treprostinil and its pharmaceutically acceptable salts.

It is a further object of the present invention to provide novel intermediates useful in the synthesis of treprostinil.

### SUMMARY OF THE INVENTION

From one aspect, the present invention provides a novel process for synthesizing treprostinil and its salts utilizing a Pauson-Khand cyclization reaction, in which the phenolic functional group is protected with p-methoxybenzyl protecting group (PMB). This choice of PMB as protecting group and more specifically the methoxy substituent on the protecting group, confers on the tricyclic intermediate compound different electronic properties and affinity to chromatographic stationary phase. This strategy leads to enhanced chromatographic properties in the preceding intermediates and allows isolation and purification of intermediates and final product.

In addition, the PMB group provides greater flexibility in its removal, allowing it to be selectively removed without removal of other protecting groups at other positions on the molecular structure, if desired, or to be removed along with removal of other protecting groups such as Bn in a single step, to reduce the overall number of process steps with resulting cost reductions. There are many processes effective for removing PMB, allowing the operator to choose one such process, based on the nature of the other protecting groups present. Conversely, the PMB protecting group can be retained while such other, different protecting groups are removed. Moreover, the PMB group also contains a chromophore, allowing assessment of purity of intermediates by HPLC methodology.

The novel process according to the invention is robust and amenable to scale up in industrial settings. It offers the advantages of cost efficiency and process simplicity. The treprostinil that can be produced by the process of the invention is of pharmaceutically acceptable quality.

Thus according to a first aspect, the present invention provides a process of preparing a substituted tricyclic enone compound useful in preparing treprostinil, the enone compound corresponding to formula 17a:

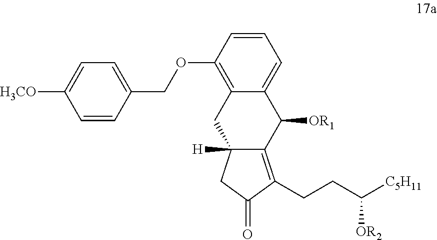

where R₁ and R₂ are independently selected alcohol protecting groups, which includes a step of subjecting an alkene-substituted, alkyne-substituted benzene corresponding to formula 16a:

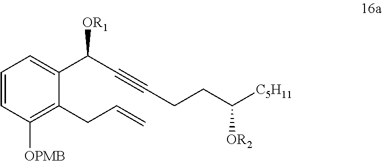

where R₁ and R₂ are independently selected alcohol protecting groups, to intramolecular cyclization with carbon monoxide.

According to another, more specific aspect of the invention, there is provided a process of preparation of treprostinil or pharmaceutically acceptable salts thereof, of formula:

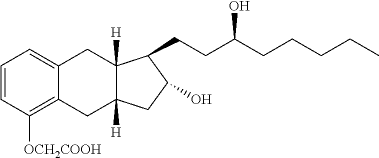

or pharmaceutically acceptable salts thereof, which comprises:

(a) derivatizing m-hydroxybenzaldehyde with an allyl halide, to form an oxyalkene-substituted benzaldehyde of formula 1a:

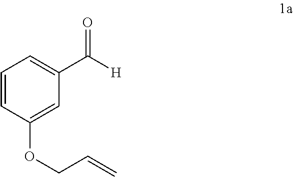

(b) subjecting the substituted benzaldehyde of formula 1a to Claisen rearrangement to form the m-hydroxy-substituted benzaldehyde of formula 1b:

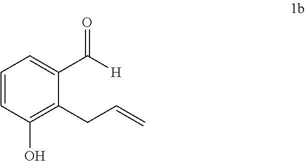

(c) reacting compound 1b with a p-methoxybenzyl halide, to form a substituted benzaldehyde of formula 11:

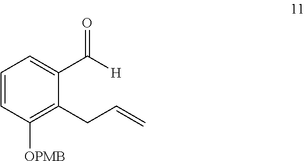

(d) reacting the protected benzaldehyde of formula 11 with a 5-oxy-substituted decan-1,2-yne of formula 12a:

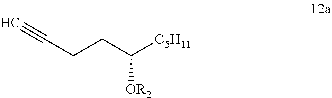

where R₂ is H or an alcohol protecting group, to yield the compound of formula 13a:

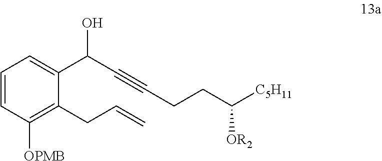

(e) oxidizing the compound of formula 13a to a compound of formula 14a:

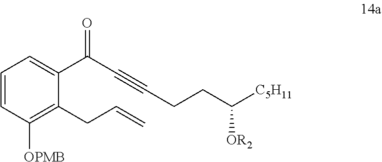

(f) chirally reducing the compound of formula 14a to a compound of formula 15a:

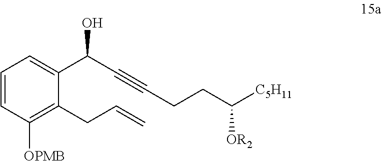

(g) protecting the compound of formula 15a to yield a compound of formula 16a:

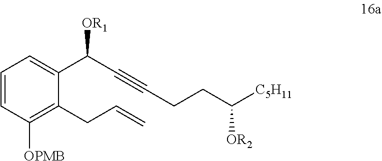

in which R₁, independently of R₂, is an alcohol protecting group;

(h) intra-molecularly cyclizing the compound of formula 16a to obtain a tricyclic enone compound of formula 17a:

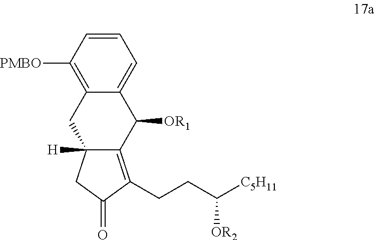

(i) converting the tricyclic enone of formula 17a to a tricyclic hydroxyl compound of formula 20:

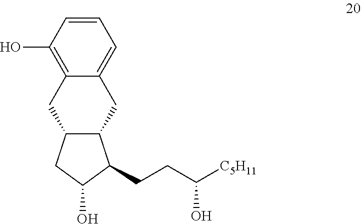

(j) alkylating the compound of formula 20 to yield a compound of formula 22:

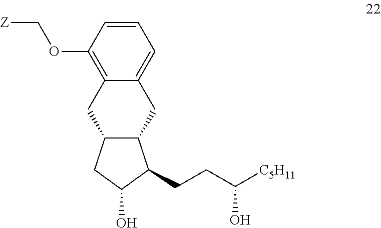

where Z is carboxyl group or a derivative thereof; and,

(k) converting the compound of formula 22 to treprostinil, followed by optional conversion to a pharmaceutically acceptable salt thereof.

### BRIEF REFERENCE TO THE DRAWING

FIG. 1 of the accompanying drawings is a chemical synthesis scheme illustrating preparation of treprostinil, via intermediates in accordance with an embodiment of the present invention.

### DESCRIPTION OF THE PREFERRED EMBODIMENTS

In the preferred process of the invention, carbon monoxide for the intramolecular cyclization reaction (the modified Pauson-Khand enyne cyclization) is provided in the form of a Group VIII transition metal-CO complex, where the transition metal is, e.g. cobalt, ruthenium, rhodium or iridium. Most preferred are cobalt-CO complexes such as cobalt octacarbonyl, Co₂(CO)₈. This procedure is known in general terms. In accordance with preferred embodiments of the invention, however, the chiral derivative 16 is protected with PMB at the phenol position, and something different, for example benzyl or TBS, at the side chain positions. Subjection of this derivative, so protected, to enyne cyclization forms the carbon skeleton of treprostinil with appropriate chirality induced. Whilst the PMB group can be installed under conditions similar to those used for benzyl, or TBS and removed under a number of comparable conditions, it also possesses the capacity to be removed under very different conditions from those effective for benzyl or TBS. Substituting the substrate with comparable yet orthogonal protecting groups, such as PMB at the phenol and benzyl at the side chain, allows the greatest scope for their differentiation. This in turn leads to wide scope to obtain material of high purity, with high yield and throughput.

It is also to be noted that the process of the present invention, in its preferred embodiments, utilizes a combination of the protecting group choice and reaction sequence that leads unexpectedly to isolatable intermediates, some of which are crystalline and so do not require purifications beyond simple re-crystallizations, and others which, although not crystalline, have improved properties rendering them readily purified by chromatography. These features are advantageous from a cost reduction standpoint. In contrast, the intermediates in the aforementioned prior art syntheses are not crystalline and therefore not amenable to purification. They require an increased number of deprotection steps, with added complications and reduced cost efficiency of those processes.

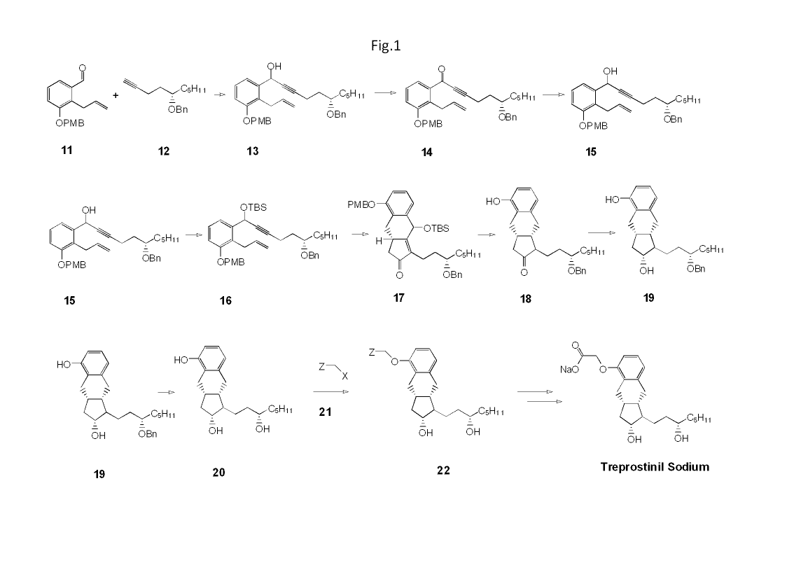

As shown in FIG. 1, the protected compound 13 can be prepared by reacting the protected benzaldehyde 11, with a substituted 1,2-alkyne 12. The illustrated protecting group Bn in compound 12 can be replaced with other suitable alcohol protecting groups such as TBDMS, etc. This reaction is known in general terms, and can be conducted by reaction in the presence of an alkali metal alkyl compound such as butyl lithium, in anhydrous organic solvent such as tetrahydrofuran. The mixture can be extracted by treatment with an aqueous salt solution, and the product recovered from the organic phase.

The protected benzaldehyde 11 shown in FIG. 1 is conveniently and preferably prepared by a modified Claisen rearrangement, using m-hydroxybenzaldehyde, a readily available commodity chemical as starting material, by a process described in companion application Serial no. NYA filed on even date herewith under the title “Protected Aldehydes for Use as Intermediates in Chemical Syntheses, and Processes for their Preparation”, the disclosure of which is incorporated herein in its entirety.

Compound 13 is next converted to its oxo analog, compound 14, by reaction with pyridinium chlorochromate (PCC) in solution in an aliphatic solvent such as dichloromethane, or by Swern like oxidation. The oxo group so formed is then reduced and further protected, e.g. with TBS by reaction with t-butylsilyl chloride, or with TBDMS by reaction with t-butyldimethylsilyl chloride, in solution in the presence of imidazole, to form compound 16. The protected compound 16, {(1R, 6S)-1-[-2-allyl-3-(4-methoxy-benzyloxy)-phenyl]-6-benzyloxy-undec-2-ynyloxy}-tert-butyl-dimethyl-silane, is recovered, dried and purified, and then subjected to modified Paulson-Khand enyne cyclization, as described above. A preferred reagent is dicobaltoctacarbonyl, and the reaction suitably takes place at room temperature in solution in a polar organic solvent such as dichloromethane. The resulting product 17, (4R,9aS)-3-((S)-3-benzyloxy-octyl)-4-(tert-butyl-dimethyl-silanyloxy)-8-(4-methoxy-benzyloxy)-1,4,9,9a-tetrahydro-cyclopenta[b]napthalen-2-one, can be recovered by filtration, and purified by column chromatography.

The remaining steps in the process according to this embodiment of the invention are removal of the various protecting groups, and are generally within the skill of the art. It is however to be noted that the removal of the silyl protecting group and removal of the PMB protecting group can be accomplished in a single reaction step, e.g. by hydrogenation over a metal catalyst such as palladium/carbon, thereby simplifying the process and reducing the overall costs. Additionally, the alkylation step (j) of the above process can be conducted using common alkylating agents such as alkyl halocarbonates, nitriles, amides, etc., generally meeting the formula Z—CH₂—X where Z is a carboxyl group or a derivative of carboxyl group such as nitrile, amide etc. and X is halo, nitrile, amide or the like group reactive with hydroxyl.

The invention is further described, for illustrative but non-limiting purposes, in the following specific examples.

### EXAMPLE 1

### Preparation of 3-Allyloxybenzaldehyde

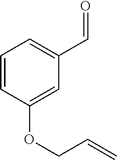

In a 1 L round bottomed flask equipped with mechanical stirrer, reflux condenser and thermometer were added 400 mL ethanol, 59.63 g of 3-hydroxybenzaldehyde (0.49 moles,1 eq.), 7.3 g of sodium iodide (48 mmol, 0.1 eq.), 120.98 g of allyl bromide (0.59 moles,1.2 eq.) and 101.6 g of potassium carbonate (0.74 moles,1.25 eq.). The reaction mixture was heated to reflux and heating continued for three hours. Heating was then discontinued and the reaction was allowed to cool to room temperature. The mixture was then filtered through a Hyflosupercel pad and ethanol was removed by rotary evaporation. The residual oil was then taken up in 500 mL of MTBE and the organic phase washed sequentially with 10% aqueous sodium hydroxide, water and brine. After drying over sodium sulfate, filtration and rotary evaporation of solvent 79.7 g of a yellow oil of 3-allyloxybenzaldehyde (quantitative yield) was obtained.

### EXAMPLE 2

### Preparation of 2-allyl-3-hydroxy-benzaldehyde

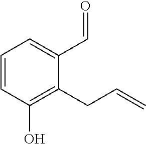

In a 500 ml three-necked Morton flask equipped with mechanical stirrer, thermometer and reflux condenser was added 100 g of 3-allyloxybenzaldehyde (0.62 moles,1 eq.) and 150 g of cis/trans decalin (1.5 vol). The mixture was purged with nitrogen and then heated to a reflux temperature of 217° C. The reaction was maintained at this reflux temperature for seven hours then cooled and added of 231 mL of toluene. The reaction mixture was then allowed to cool to room temperature. After stirring for 18 hours and further cooling to 0-5° C. for 1-2 hours, reaction mixture was filtered and the cake washed with 200 mL of heptane. The wet cake was stirred in 200 mL of heptane for 1-2 hours at room temperature. After filtration and drying of the cake at 40° C., 54.27 g of crude 2-allyl-3-hydroxy-benzaldehyde were obtained. This represents a recovery of 82% of the available 2-allyl product produced by the Claisen rearrangement.

### EXAMPLE 3

### Preparation of 2-allyl-3-(4-methoxy-benzyloxy)-benzaldehyde (Compound 11, FIG. 1)

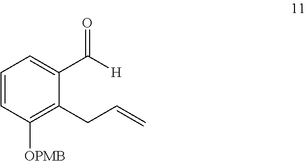

In a 1 L three necked round bottom flask equipped with a mechanical stirrer, thermometer and reflux condenser was added 300 mL acetone, 23.19 g of 2-allyl-3hydroxybenzaldehyde (0.143 mole, 1 eq.), 2.13 g of sodium iodide (14 mmol., 0.1 eq), 39.52 g of potassium carbonate (28.6 mmol., 2 eq.) and 22.39 g of p-methoxybenzyl chloride (14.3 mmol., 1 eq.). The reaction mixture was heated to reflux for 4 hours. After cooling reaction mixture to room temperature, the reaction mixture was filtered through a bed of Hyflosupercel and the solvent removed by rotary evaporation. The residual dark oil was taken up in 200 mL of toluene and washed sequentially with 10% aqueous sodium hydroxide, water and brine. The organic phase was dried over sodium sulfate and decolourized with 5 g Darco G60. After filtration through a Celite pad, the solvent was removed by rotary evaporation to give 35.5 g of oil which was then recrystallized from 175 mL of hot IPA. After cooling to room temperature and further cooling to 0-5° C. for 1-2 hours, the solids were filtered and washed with IPA to afford after drying at 40° C., 24.74 g (61%) of 2-allyl-3-(4-methoxy-benzyloxy)-benzaldehyde as an off-white solid.

### EXAMPLE 4

### Preparation of (S)-1-[-2-Allyl-3-(4-methoxy-benzyloxy)-phenyl]-6-benzyloxy-undec-2-yn-1-ol (Compound 13, FIG. 1)

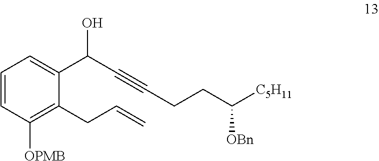

n-butyllithium (3.5 mL of 2.5M in hexane; 8.75 mmol) was added to a cooled solution of ((S)-1-but-3-ynyl-hexyloxymethyl)-benzene (2 g; 8.18 mmol) in anhydrous tetrahydrofuran (9 mL). After stirring for 1-2 hours, a solution of 2-allyl-3-(4-methoxy-benzyloxy)-benzaldehyde (1.5 g; 5.31 mmol) in tetrahydrofuran (4.5 mL) was added. After stirring for 3-4 hours, saturated ammonium chloride (15 mL) was added followed by 5 mL of water. The layers were separated and aqueous layer further extracted with 1x5 mL methyl t-butylether. The combined organic layers were dried over magnesium sulfate and filtered. After solvent evaporation and purification of crude oil by column chromatography using a mixture of heptane and ethyl acetate, 2.3 g (82.1% based on aldehyde) of desired compound was obtained.

### EXAMPLE 5

### Preparation of (S)-1-[-2-Allyl-3-(4-methoxy-benzyloxy)-phenyl]-6-benzyloxy-undec-2-yn-1-one (Compound 14, FIG. 1)

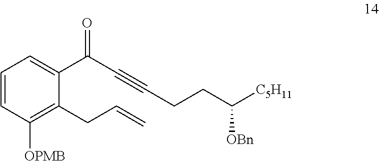

A mixture of (S)-1-[-2-Allyl-3-(4-methoxy-benzyloxy)-phenyl]-6-benzyloxy-undec-2-yn-1-ol (2.3 g; 4.37 mmol) and pyridinium chlorochromate (2.0 g; 9.28 mmol) in dichloromethane (13 mL) was stirred at room temperature for 3-4 hours. Celite (6.1 g) was added to the mixture, followed by heptane (15 mL) and the resulting mixture was stirred for 1 hour. After filtration and evaporation, the crude oil was purified by column chromatography using a mixture of heptane and ethyl acetate to give the desired compound as an oil (1.9 g; 83%).

### EXAMPLE 6

### Preparation of (1R, 6S)-1-[-2-allyl-3-(4-methoxy-benzyloxy)-phenyl]-6-benzyloxy-undec-2-yn-1-ol (Compound 15, FIG. 1)

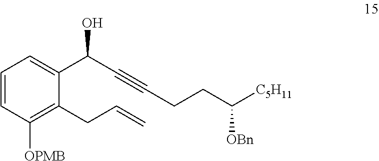

A solution of (R)-methyl oxazaborolidine (3.6 mL of 1M in toluene; 3.6 mmoL) was added to a solution of (S)-1-[-2-Allyl-3-(4-methoxy-benzyloxy)-phenyl]-6-benzyloxy-undec-2-yn-1-one (1.9 g; 3.6 mmoL) in anhydrous tetrahydrofuran (13 mL) at -25° C. to −30° C., followed by a solution of borane dimethylsulfide complex (7.0 mL of 2M in toluene; 14 mmoL). After 1-2 hours of stirring, methanol (2 mL) was added and reaction mixture was allowed to warm to 10° C. Water (20 mL) was then added. After stirring, the layers were separated and the aqueous layer extracted with 1×10 mL toluene. The combined organic layers were dried over magnesium sulfate, filtered and concentrated under vacuum to give a crude oil that was purified by column chromatography using a mixture of heptane and ethyl acetate. (1.5 g; 78.5%)

### EXAMPLE 7

### Preparation of {(1R, 6S)-1-[-2-Allyl-3-(4-methoxy-benzyloxy)-phenyl]-6-benzyloxy-undec-2-ynyloxy}-tert-butyl-dimethyl-silane (Compound 16, FIG. 1)

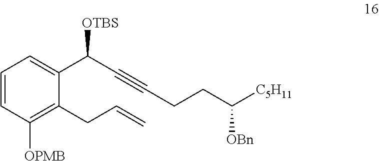

A mixture of (1R, 6S)-1-[-2-Allyl-3-(4-methoxy-benzyloxy)-phenyl]-6-benzyloxy-undec-2-yn-1-ol (1.5 g; 2.9 mmoL), t-butyldimethylsilylchloride (0.6 g; 3.9 mmoL) and imidazole (0.4 g; 5.7 mmoL) in dichloromethane (15 mL) was stirred at room temperature overnight. Water (15 mL) was then added and reaction mixture stirred. The layers were separated and aqueous layer further extracted with 1×5 mL dichloromethane. The combined organic layers were dried over magnesium sulfate, filtered and concentrated under vacuum to give an oil (1.7 g). Purification by column chromatography using a mixture of heptane and ethyl acetate gave the desired Pauson-Khand cyclization substrate (1.5 g; 82.2%).

### EXAMPLE 8

### Preparation of (4R,9aS)-3-((S)-3-Benzyloxy-octyl)-4-(tert-butyl-dimethyl-silanyloxy)-8-(4-methoxy-benzyloxy)-1,4,9,9a-tetrahydro-cyclopenta[b]napthalen-2-one (Compound 17, FIG. 1)

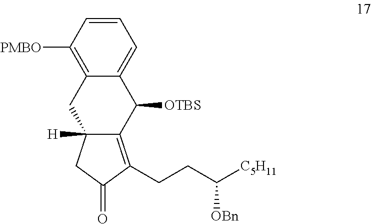

Cobalt octacarbonyl (0.62 g; 1.83) was added to a solution of {(1R, 6S)-1-[-2-Allyl-3-(4-methoxy-benzyloxy)-phenyl]-6-benzyloxy-undec-2-ynyloxy}-tert-butyl-dimethyl-silane (1.1 g; 1.72 mmoL) in dichloromethane (10 mL; degassed by vacuum) at room temperature under nitrogen. After stirring for 3-4 hours, acetonitrile (5 mL; degassed by vacuum) was added to the reaction mixture and dichloromethane was distilled off. More degassed acetonitrile (5 mL) was added to the reaction mixture and additional dichloromethane was distilled. After cooling reaction mixture to room temperature, Celite (1.3 g) was added and stirring continued for 1-2 hours. Methyl t-butylether (5 mL) was then added to the mixture and reaction mixture filtered over Celite (1.1 g). The filtrate was concentrated under vacuum to an oil that was purified by column chromatography using 10% methyl t-butylether in hexane to give the desired compound as an oil (0.96 g; 83.6%).

### EXAMPLE 9

### Preparation of (S)-1-(S)-3-Benzyloxy-octyl)-5-hydroxy-1,3,3a,4,9,9a-hexahydro-benz[f]inden-2-one (Compound 18, FIG. 1)

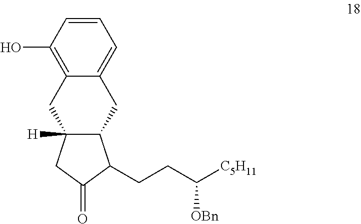

A mixture of (4R,9aS)-3-((S)-3-Benzyloxy-octyl)-4-(tert-butyl-dimethyl-silanyloxy)-8-(4-methoxy-benzyloxy)-1,4,9,9a-tetrahydro-cyclopenta[b]napthalen-2-one (0.94 g; 1.41 mmoL), 10% Pd/C (192 mg; 20.4 wt %) and potassium carbonate (47 mg; 5 wt %) in anhydrous ethanol(10 mL) stirred at room temperature under hydrogen pressure. After 16-18 hours, reaction mixture was filtered over Celite, cake washed with methyl t-butylether and solution concentrated under vacuum. Purification by column chromatography using a mixture of heptane and ethyl acetate, gave an oil (401 mg; 67.7%) as a mixture of epimers.

### EXAMPLE 10

### Preparation of (1R,2R,3aS,9aS)-1-(S)-3-Benzyloxy-octyl)-2,3,3a,4,9,9a-hexahydro-1H-cyclopenta[b]naphthalene-2,5-diol (Compound 19, FIG. 1)

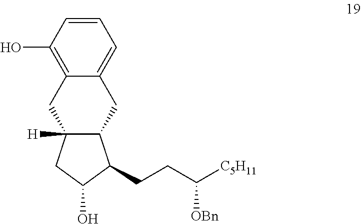

20% Aqueous sodium hydroxide (1.9 mL; 9.5 mmoL) was added to a solution of (S)-1-((S)-3-Benzyloxy-octyl)-5-hydroxy-1,3,3a,4,9,9a-hexahydro-benz[f]inden-2-one (401 mg; 0.95 mmoL) in anhydrous ethanol (3.5 mL). Sodium borohydride (41.7 mg; 1.1 mmoL) was added and reaction mixture stirred for 3-4 hours. A saturated ammonium chloride was added and mixture extracted with methyl t-butylether. Aqueous layer was diluted with 1M aqueous hydrochloric acid and extracted with methyl t-butylether. Combined organic layers were dried over magnesium sulfate, filtered and concentrated to give an oil (0.34 g) that was used without purification in the next step.

### EXAMPLE 11

### Preparation of (1R,2R,3aS,9aS)-1-(S)-3-Hydroxy-octyl)-2,3,3a,4,9,9a-hexahydro-1H-cyclopenta[b]naphthalene-2,5-diol (Compound 20, FIG. 1)

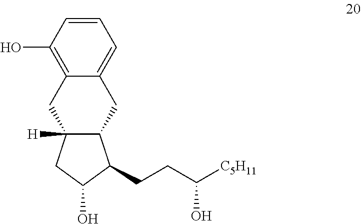

A mixture of (1R,2R,3aS,9aS)-1-((S)-3-Benzyloxy-octyl)-2,3,3a,4,9,9a-hexahydro-1H-cyclopenta[b]naphthalene-2,5-diol (0.25 g; crude material) and 10% Pd—C (51 mg) in glacial acetic acid (5 mL) was stirred under hydrogen pressure for 3-6 hours. Reaction mixture was then filtered over Celite and cake washed with ethyl acetate. The filtrate was concentrated under vacuum using heptane to give a crude material (0.2 g) that was used in the next step.

### EXAMPLE 12

### Preparation of [(1R,2R,3aS,9aS)-2-Hydroxy-1-(S)-3-hydroxy-octyl)-2,3,3a,4,9,9a-hexahydro-1H-cyclopenta[b]naphthalene-5-yloxyl-acetic acid, ethyl ester

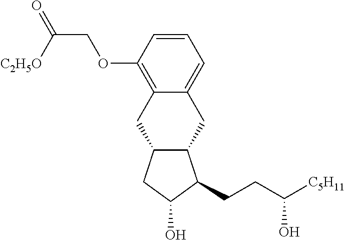

Ethyl bromoacetate (151 mg; 0.90 mmoL) was added to a mixture of (1R,2R,3aS,9aS)-1-((S)-3-Hydroxy-octyl)-2,3,3a,4,9,9a-hexahydro-1H-cyclopenta[b]naphthalene-2,5-diol (185 mg; 0.56 mmoL) and potassium carbonate (161 mg; 1.2 mmoL) in anhydrous ethanol (5 mL). After stirring at room temperature for 1-2 hours, the mixture was refluxed for 1-2 hours and tetra butylammonium iodide (21 mg) was added. The mixture was refluxed for another 3-4 hours, cooled to room temperature and filtered. The filtrate was concentrated under vacuum to give an oil that was purified by column chromatography using a mixture of heptane and ethyl acetate to give the desired compound as an oil (159 mg; 68.0%).

### EXAMPLE 13

### Preparation of [(1R,2R,3aS,9aS)-2-Hydroxy-1-(S)-3-hydroxy-octyl)-2,3,3a,4,9,9a-hexahydro-1H-cyclopenta[b]naphthalene-5-yloxyl-acetic acid, methyl ester

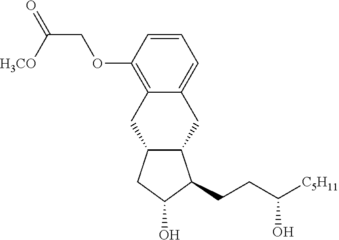

A mixture of (1R,2R,3aS,9aS)-1-((S)-3-Hydroxy-octyl)-2,3,3a,4,9,9a-hexahydro-1H-cyclopenta[b]naphthalene-2,5-diol (3.8 g; 11.3 mmoL), methyl bromoacetate (2.2 g; 14.1 mmoL) and potassium carbonate (3.1 g; 22.4 mmoL) in acetone (30 mL) was refluxed for 6.5 hours and then cooled to room temperature. The reaction mixture was filtered and cake washed with acetone. The filtrate was concentrated and dried under high vacuum to give 4.5 g of crude material that was carried to the next step.

### EXAMPLE 14

### Preparation of [(1R,2R,3aS,9aS)-2-Hydroxy-1-((S)-3-hydroxy-octyl)-2,3,3a,4,9,9a-hexahydro-1H-cyclopenta[b]naphthalene-5-yloxyl-acetic acid

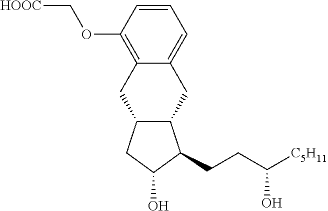

[(1R,2R,3aS,9aS)-2-Hydroxy-1-((S)-3-hydroxy-octyl)-2,3,3a,4,9,9a-hexahydro-1H-cyclopenta[b]naphthalene-5-yloxy]-acetic acid, ethyl ester (142 mg; 0.34 mmoL) was dissolved in anhydrous ethanol (1 mL). Aqueous 20% sodium hydroxide (0.1 mL; ˜20 mg; ˜0.5 mmoL) was added and mixture stirred for 1-2 hours. 1M aqueous hydrochloric acid (1 mL) was added followed by water and methyl t-butylether. The layers were separated and the aqueous layer further extracted with methyl t-butylether. The combined organic layers were dried over magnesium sulfate, filtered and concentrated under vacuum, to give treprostinil as a solid.

### EXAMPLE 15

### Preparation of [(1R,2R,3aS,9aS)-2-Hydroxy-1-((S)-3-hydroxy-octyl)-2,3,3a,4,9,9a-hexahydro-1H-cyclopenta[b]naphthalene-5-yloxyl-acetic acid, sodium salt

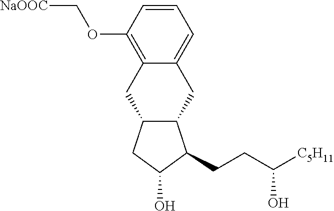

[(1R,2R,3aS,9aS)-2-Hydroxy-1-((S)-3-hydroxy-octyl)-2,3,3a,4,9,9a-hexahydro-1 H-cyclopenta[b]naphthalene-5-yloxy]-acetic acid, (513 mg, 1.3 mmoL) was dissolved in anhydrous ethanol (3 mL). An ethanolic solution of sodium hydroxide (4.6 mL of 0.28M; 1.3 mmoL) was added and the reaction stirred for 1-2 hours at room temperature. Toluene (5 mL) was added and the solution was concentrated under vacuo to give a solid residue. To this crude material was then added ethyl acetate (5 mL) and the suspension was stirred for 1-2 hours at room temperature. After filtration, the cake was washed with ethyl acetate and dried under vacuum.

What is claimed is:

1. A process of preparing a substituted tricyclic enone compound useful in preparing treprostinil, the enone compound corresponding to formula 17a: 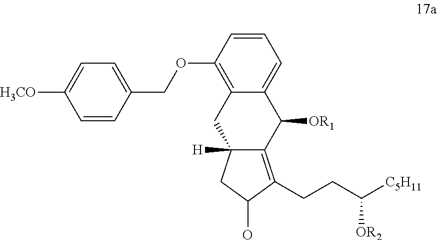
where R₁ and R₂ are independently selected alcohol protecting groups, which includes a step of subjecting an alkene-substituted, alkyne-substituted benzene corresponding to formula 16a 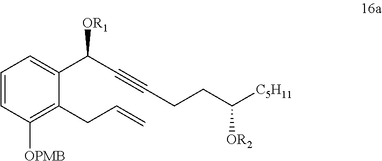
where R₁ and R₂ are independently selected alcohol protecting groups, to intramolecular cyclization with carbon monoxide.

2. The process of claim 1 wherein the carbon monoxide for intramolecular cyclization is used in the form of a Group VIII transition metal-carbon monoxide complex

3. The process of claim 1 wherein the carbon monoxide for intramolecular cyclization is used in the form of a cobalt-carbon monoxide complex.

4. The process of claim 3 wherein the alkene-substituted, alkyne-substituted benzene compound of formula 13a is prepared by reacting an alkene-substituted benzaldehyde of formula 11: 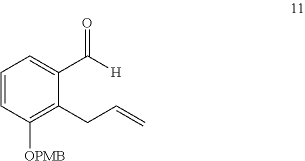
with a substituted 1,2-alkyne of formula 12a: 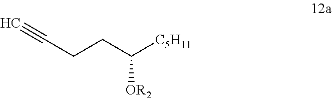
in which R₂ has the meaning given in claim 1.

5. The process of claim 4 wherein the benzaldehyde of formula 11 is prepared by modified Claisen rearrangement of an O-allyl-substituted benzaldehyde of formula 1a: 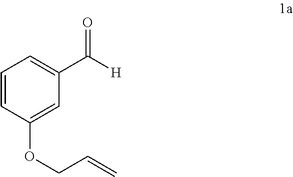
followed by reaction with p-methoxybenzyl halide to protect the resultant meta-phenolic group.

6. The process of any preceding claim including the additional, subsequent step of removing the p-methoxy benzyl protecting group and the group R₁.

7. A process of preparation of treprostinil or pharmaceutically acceptable salts thereof, of formula: 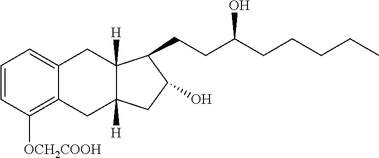
or pharmaceutically acceptable salts thereof, which comprises:
(a) derivatizing m-hydroxybenzaldehyde with an allyl halide, to form an oxyalkene-substituted benzaldehyde of formula 1a: 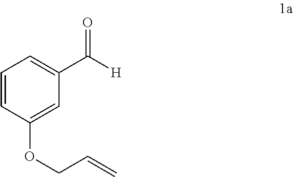
(b) subjecting the substituted benzaldehyde of formula 1a to Claisen rearrangement to form the m-hydroxy-substituted benzaldehyde of formula 1b: 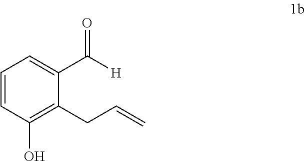
(c) reacting compound 1b with a p-methoxybenzyl halide, to form a substituted benzaldehyde of formula 11: 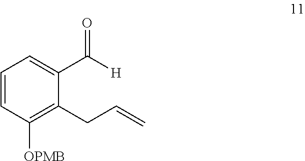
(d) reacting the protected benzaldehyde of formula 11 with a 5-oxy-substituted decan-1,2-yne of formula 12a: 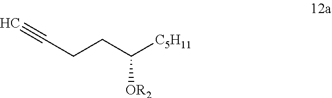
where R₂ is H or an alcohol protecting group, to yield the compound of formula 13a: 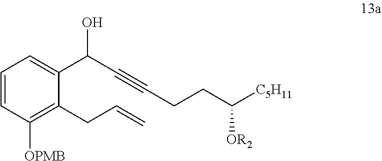
(e) oxidizing the compound of formula 13a to a compound of formula 14a: 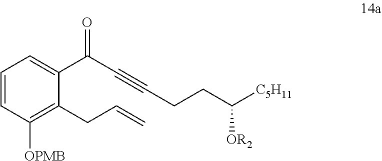
(f) chirally reducing the compound of formula 14a to a compound of formula 15a: 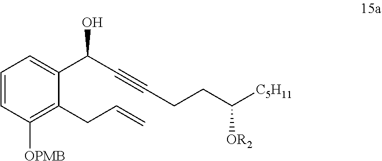
(g) protecting the compound of formula 15a to yield a compound of formula 16a: 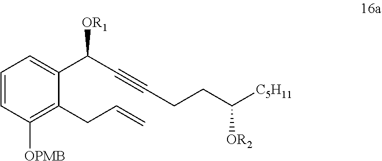
in which R₁, independently of R₂, is an alcohol protecting group;
(h) intra-molecularly cyclizing the compound of formula 16a to obtain a tricyclic enone compound of formula 17a: 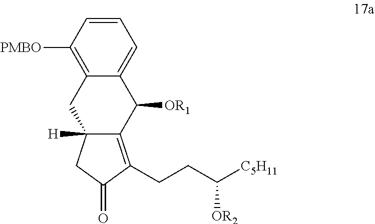
(i) converting the tricyclic enone of formula 17a to a tricyclic hydroxyl compound of formula 20: 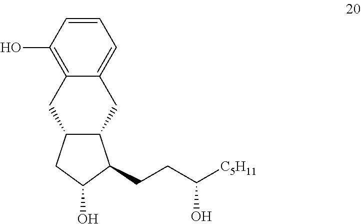
(j) alkylating the compound of formula 20 to yield a compound of formula 22: 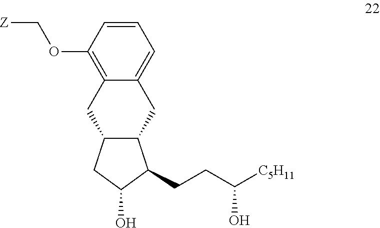
where Z is carboxyl group or a derivative thereof;
and (k) converting the compound of formula 22 to treprostinil, followed by optional conversion to a pharmaceutically acceptable salt thereof.

8. The process of claim 7 including the additional, final step of converting the treprostinil so formed to its sodium salt.

9. The process of claim 7 or claim 8 wherein the alkylation step (j) is conducted using an alkyl bromoalkanoate.

10. A substituted tricyclic enone compound useful in the synthesis of pharmaceutically active prostacyclin derivatives, corresponding to the formula 17a: 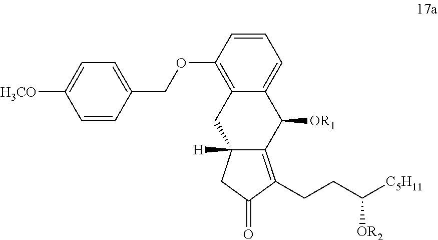
wherein R₁ and R₂ are independently selected from hydrogen and alcohol protecting groups.

11. A substituted chiral compound of formula 16a 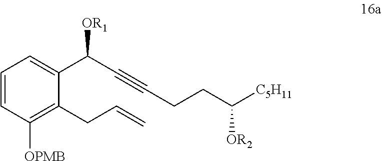
wherein R₁, independently of R₂, is an alcohol protecting group.

12. A substituted compound of formula 15a 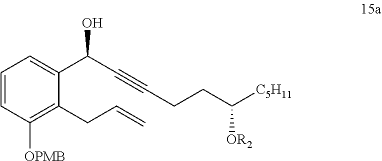
wherein R₂ is an alcohol protecting group.

13. A substituted compound of formula 14a 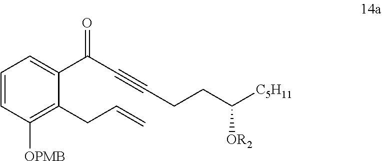
wherein R₂ is alcohol protecting group.

14. A substituted compound of formula 13a 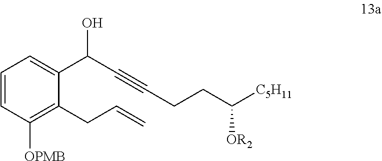
wherein R₂ is an alcohol protecting groups.
